(19) United States
(12) Patent Application Publication
(10) Pub. No.: US 2010/0279951 A1
(43) Pub. Date: Nov. 4, 2010

(54) METHOD OF TREATING ALLERGIC CONJUNCTIVITIS WITH CYCLOSPORIN COMPOSITIONS

(76) Inventors: Aileen Morgan, Rancho Santa Margarita, CA (US); Rhett M. Schiffman, Laguna Beach, CA (US)

Correspondence Address: ALLERGAN, INC., 2525 DUPONT DRIVE, T2-7H, IRVINE, CA, 92612-1599, US

(21) Appl. No.: 12/771,952

(22) Filed: Apr. 30, 2010

Related U.S. Application Data
(60) Provisional application No. 61/174,824, filed on May 1, 2009.

(51) Int. Cl. A61K 38/13; A61P 27/02

(52) U.S. Cl. 514/20.5

(57) ABSTRACT

Disclosed herein is a method of treating allergic conjunctivitis, the method comprising the step of topically administering to an eye affected with such a condition a composition comprising cyclosporin, at a concentration between about 0.001% (w/v) to about 0.01% (w/v).

### CROSS-REFERENCES

This application claims priority to U.S. Provisional Patent Application No. 61/174,824 filed on May 1, 2009, the entire disclosure of which is incorporated herein by this specific reference.

### INTRODUCTION

Disclosed herein is a method of treating allergic conjunctivitis, the method comprising topically administering to an eye affected with such a condition a composition containing cyclosporin having a concentration between about 0.001% (w/v) to about 0.01% (w/v).

### DETAILED DESCRIPTION OF THE INVENTION

### Allergic Conjunctivitis

Allergic conjunctivitis is an inflammation of the conjunctiva resulting from hypersensitivity to one or more allergens. It may be acute, intermittent, or chronic.

In one embodiment, the method of the invention may be used to treat seasonal allergic conjunctivitis. The allergens responsible for seasonal allergic conjunctivitis is usually the pollen of trees, grasses, or weeds; hence it is sometimes called hay fever conjunctivitis, being associated with the same allergens that cause that hay fever. The course of the disease tends to follow the prevalence of these allergens in the air. As their numbers vary according to the time of season, so too does the severity of the conjunctivitis associated with it.

In another embodiment, the method of the invention may be used to treat perennial allergic conjunctivitis. The allergens responsible for perennial allergic conjunctivitis are usually dust mites, animal dander, and other allergens the prevalence of which does not vary according to the season. A hypersensitivity to food is sometimes the cause. Hence, the condition may be chronic, or otherwise occur independently of the time of season.

Symptoms of seasonal and perennial allergic conjunctivitis include, in addition to inflammation of the conjunctiva, lacrimation, tearing, conjunctival vascular dilation, itching, papillary hyperlasia, chemosis, eyelid edema, and discharge from the eye. The discharge may form a crust over the eyes after a night's sleep.

In another embodiment, the method of the invention may be used to treat atopic keratoconjunctivitis. Atopic keratoconjunctivitis is a chronic, severe form of allergic conjunctivitis that often leads to visual impairment. Symptoms include itching, burning, pain, redness, foreign body sensation, light sensitivity and blurry vision. There is often a discharge, especially on awakening from a night's sleep; the discharge may be stringy, ropy, and mucoid. The lower conjunctiva is often more prominently affected than the upper conjunctiva. The conjunctiva may range from pale, edematous, and featureless to having the characteristics of advanced disease, including papillary hypertrophy, subepithelial fibrosis, formix foreshortening, trichiasis, entropion, and madurosis. In some patients the disease progresses to punctate epithelial erosions, corneal neovascularization, and other features of keratopathy which may impair vision. There is typically goblet cell proliferation in the conjunctiva, epithelial pseudotubular formation, and an increased number of degranulating eosinophils and mast cells in the epithelium. CD25+T lymphocytes, macrophages, and dendritic cells (HLA-DR⁺, HLA-CD1+) are significantly elevated in the substantia propria.

In another embodiment, the method of the invention may be used to treat vernal keratoconjunctivitis. Like atopic keratoconjunctivitis, vernal keratoconjunctivitis is a severe form of allergic conjunctivitis, but it tends to affect the upper conjunctiva more prominently than the lower. It occurs in two forms. In the palpebral form, square, hard, flattened, closely packed papillae are present; in the bulbar (limbal) form, the circumcorneal conjunctiva becomes hypertrophied and grayish. Both forms are often accompanied by a mucoid discharge. Corneal epithelium loss may occur, accompanied by pain and photophobia, as may central corneal plaques and Trantas' dots.

Topical cyclosporin has been used to treat atopic keratoconjunctivitis and vernal keratoconjunctivitis. Akpek et al. report using Restasis®, a formulation of cyclosporin 0.05% (Allergan, Irvine, Calif.), to treat atopic keratoconjunctivitis in a trial involving twenty subjects, half of whom received Restasis® and the other placebo. Opthalmology, 111(3): 476-482 (2004). Bleik and Tabbara, Opthalmology 98:1679-1684 (1991), and Mendicute et al., Eye, 11(1): 75-78 (1997), report using a topical formulation of cyclosporin 2.0% to treat patients with vernal keratoconjunctivitis.

The inventors disclose here a formulation and method that enables one to treat allergic conjunctivitis, including its most severe forms (atopic keratoconjunctivitis and vernal keratoconjunctivitis), using cyclosporin compositions at concentrations far less than that reported in the medical literature.

### Cyclosporin

Cyclosporins are a group of nonpolar cyclic oligopeptides with known immunosuppressant activity. Cyclosporin A, along with several other minor metabolites, as well as cyclosporin B, C, D, E, F, G, H, I, J, K, L, M, N, O, P, Q, R, S, T, U, V, W, X, Y and Z, have been identified. In addition, derivatives, salts and the like of such cyclosporins and a number of synthetic analogs have been prepared and may be useful in the present invention. The use of cyclosporin-A and cyclosporin A derivatives to treat ophthalmic conditions has been the subject of various patents, for U.S. Pat. No. 5,474,979, No. 6,254,860, and No. 6,350,442, this disclosure of each of which is incorporated in its entirety by reference.

In general, commercially available cyclosporins may contain a mixture of several individual cyclosporins which all share a cyclic peptide structure consisting of eleven amino acid residues with a total molecular weight of about 1,200, but with different substituents or configurations of some of the amino acids.

As used here, a “cyclosporin composition” includes any individual member of the cyclosporin group, salts thereof, derivatives thereof, analogs thereof and mixtures thereof, as well as mixtures of two or more individual cyclosporins salts thereof, derivatives thereof, analogs thereof and mixtures thereof.

In one embodiment, the cyclosporin composition comprises cyclosporin A, a derivative of cyclosporin A, a salts of cyclosporin A, and/or mixtures thereof.

The chemical structure for cyclosporin A is represented by Formula 1. It has the chemical name cyclo[[(E)-(2S,3R,4R)-3-hydroxy-4-methyl-2-(methylamino)-6-octenoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl].

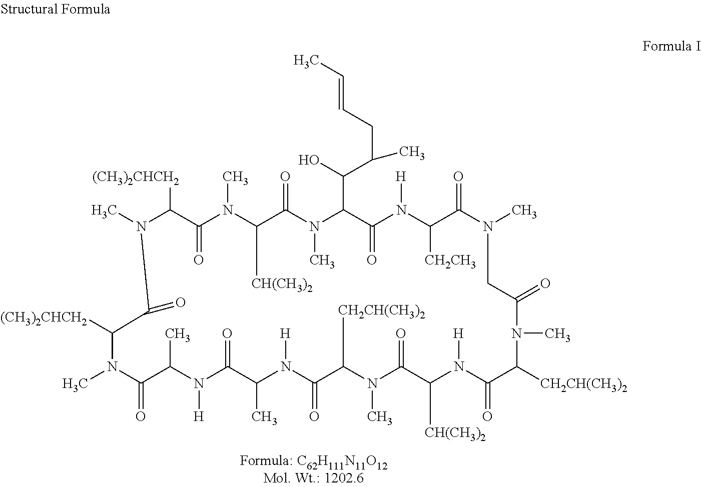

As used here, the term “derivatives” of a cyclosporin refer to compounds having structures sufficiently similar to the cyclosporin so as to function in a manner substantially similar to or substantially identical to cyclosporin A. Included, without limitation, within the useful cyclosporin A derivatives are those selected from ((R)-methylthio-Sar)³-(4′-hydroxy-MeLeu) cyclosporin A, ((R)-(Cyclo)alkylthio-Sar)³-(4′-hydroxy-MeLeu)⁴-cyclosporin A, and ((R)-(Cyclo)alkylthio-Sar)³-cyclosporin A derivatives described below.

These cyclosporin derivatives are represented by the following general formulas (II), (III), and (IV) respectively:

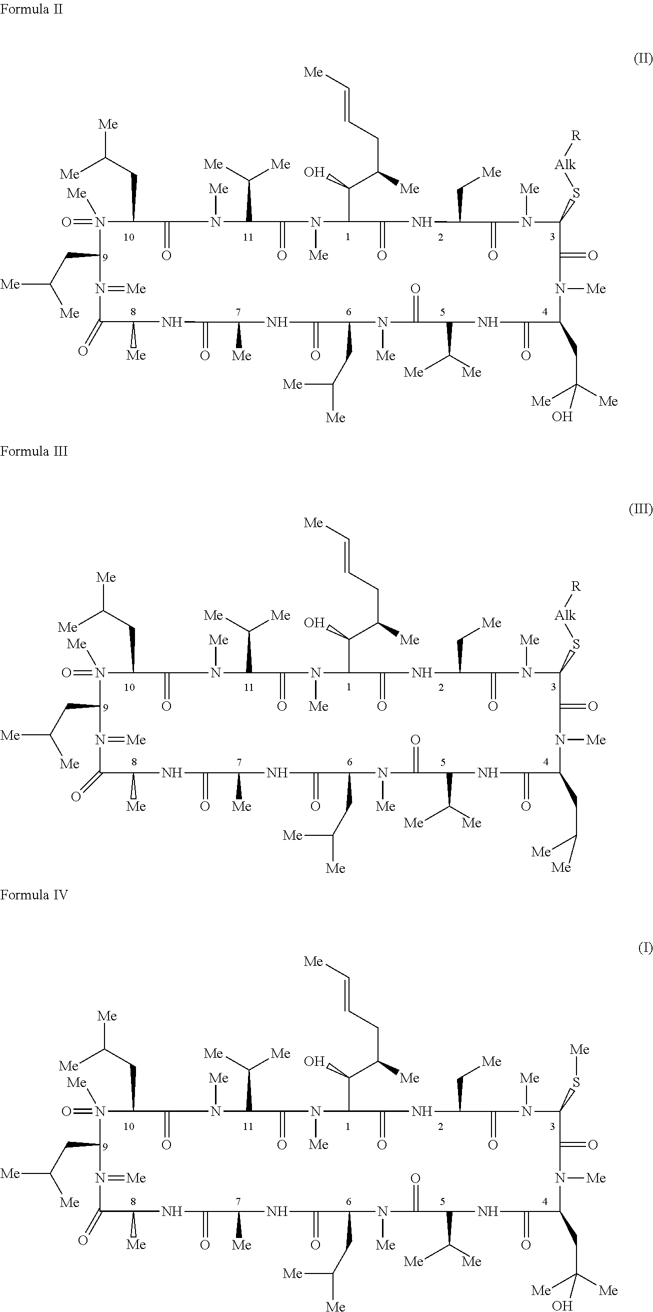

wherein Me is methyl; Alk is 2-6C alkylene or 3-6C cycloalkylene; R is OH, COOH, alkoxycarbonyl, —NR₁R₂ or N(R₃)—(CH₂)—NR₁R₂; wherein R₁, R₂ is H, alkyl, 3-6C cycloalkyl, phenyl (optionally substituted by halo, alkoxy, alkoxycarbonyl, amino, alkylamino or dialkylamino), benzyl or saturated or unsaturated heterocyclyl having 5 or 6 members and 1-3 heteroatoms; or NR₁R₂ is a 5 or 6 membered heterocycle which may contain a further N, O or S heteroatom and may be alkylated; R₃ is H or alkyl and n is 2-4; and the alkyl moieties contain 1-4C.

The cyclosporin composition contains from about 0.005% to 0.01% cyclosporin by weight of the composition. In one embodiment, the composition contains cyclosporin in an amount of about 0.01% (w/v). In another embodiment, the composition contains cyclosporin in an amount of about 0.005% (w/v). In another embodiment, the composition contains cyclosporin in an amount of about 0.0075% (w/v). In one embodiment, the composition contains 0.005% (w/v), 0.0075% (w/v), or 0.01% (w/v) cyclosporin A.

The cyclosporin composition is formulated such that it can be administered topically to the eye. Such formulations are disclosed, for example, in U.S. Pat. Nos. 5,474,979 and 7,501,393, and in U.S. Patent Application Publication No. 2005/0059583, No. 2007/0015693, No. 2007/0299004, No. 2008/0039378, No. 2008/0146497, No. 2008/0207494, and No. 2008/0207495, the disclosures of all of which are incorporated by reference.

Solutions are often prepared using a physiological saline solution as a major vehicle. Ophthalmic solutions are often maintained at a comfortable pH with an appropriate buffer system. The formulations may also contain conventional, pharmaceutically acceptable preservatives, stabilizers and surfactants.

Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed.

In another embodiment, the composition contains a preservative.

Preservatives that may be used in the pharmaceutical compositions disclosed herein include, but are not limited to, cationic preservatives such as quaternary ammonium compounds (including benzalkonium chloride, polyquad, and the like; guanidine-based preservatives (including PHMB, chlorhexidine, and the like); chlorobutanol; mercury preservatives such as thimerosal, phenylmercuric acetate, and phenylmercuric nitrate; and oxidizing preservatives such as stabilized oxychloro complexes (e.g. Purite®, a stabilized chlorine dioxide).

A surfactant may be used for assisting in dissolving an excipient or an active agent, dispersing a solid or liquid in a composition, enhancing wetting, modifying drop size, or a number of other purposes. Useful surfactants include, but are not limited to surfactants of the following classes: alcohols; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal & veg.); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants; propoxylated & ethoxylated fatty acids, alcohols, or alkyl phenols; protein-based surfactants; sarcosine derivatives; sorbitan derivatives; sucrose and glucose esters and derivatives.

In particular, ethoxylate surfactants are useful.

An ethoxylate surfactants is one that comprises the moiety —O(CH₂CH₂O)ₙ—OH, wherein n is at least about 1.

In one embodiment n is from about 1 to about 10,000.

In another embodiment, n is from 1 to about 1000.

In another embodiment, n is from about 1 to about 500.

Some ethoxylates contain one ethoxylate moiety. In other words, there is a single ethoxylate chain on each molecule.

Examples of surfactants with one ethoxylate moiety, include, but are not limited to:

Ethoxylated alcohols wherein the alcohol has a single hydroxyl unit; alkylphenol ethoxylates; ethoxylated fatty acids; fatty acid methyl ester ethoxylates; polyethylene glycols; and the like.

Ethoxylates may comprise more than one ethoxylate moiety. In other words, there may be ethoxylate moieties attached to several different parts of the molecule. Examples include, but are not limited to: block polymers; ethoxylated oils; so derivatives; sucrose and glucose ethoxylates; and the like.

Block Polymers These are polymers with the structure A-B-A′, wherein A and A′ are polyethylene chains of 1 or more ethylene units, and B is a polypropylene chain of one or more propylene units. Generally, but not necessarily, A and A′ are approximately the same length.

In one embodiment, A and A′ contain from about 2 to about 200 ethylene units.

In another embodiment, A and A′ contain from about 5 to about 100 ethylene units.

In another embodiment, A and A′ contain about 7 to about 15 ethylene units.

In another embodiment, A and A′ contain about 7, about 8, or about 12 ethylene units.

In another embodiment, B contains from about 25 to about 100 propylene units.

In another embodiment, B contains from about 30 to about 55 propylene units.

In another embodiment, B contains about 30, about 34, or about 54 propylene units.

In another embodiment, the molecular weight is from about 1000 to about 20000.

In another embodiment, the molecular weight is from about 2000 to about 10000.

In another embodiment, the molecular weight is about 2500, about 3000, about 3800, or about 8400.

These include but are not limited to:

Poloxalene: wherein A has about 12 ethylene oxide units, B has about 34 propylene oxide units, A′ has about 12 ethylene oxide units, and the average molecular weight is about 3000.
Poloxamer 182: wherein A has about 8 ethylene oxide units, B has about 30 propylene oxide units, A′ has about 8 ethylene oxide units, and the average molecular weight is about 2500
Poloxamer 188: wherein A has about 75 ethylene oxide units, B has about 30 propylene oxide units, A′ has about 75 ethylene oxide units, and the average molecular weight is about 8400.
Poloxamer 331: wherein A has about 7 ethylene oxide units, B has about 54 propylene oxide units, A′ has about 7 ethylene oxide units, and the average molecular weight is about 3800;

### Ethoxylated Alcohols

These include but are not limited to:

Ethoxylates of linear alcohols having from about 6 to about 20 carbon atoms.

In one embodiment, the linear alcohol has from about 10 to about 16 carbon atoms.

In another embodiment, n is from about 1 to about 100.

In another embodiment, n is from about 1 to about 50.

In another embodiment, n is from about 5 to about 50 ethylene oxide units.

In another embodiment, n is from about 1 to about 20 ethylene oxide units.

In another embodiment, n is from about 30 to about 50 ethylene oxide units.

### Ethoxylated Alkylphenols

These are alkylphenols that are ethoxylated, i.e. the phenolic OH is replaced with an ethoxylate moiety.

These include but are not limited to:

octylphenol ethoxylate, i.e. C₈H₁₇Ph(OCH₂CH₂O)ₙH.
nonylphenol ethoxylate, i.e. C₉H₁₉Ph(OCH₂CH₂O)ₙH.
alkyphenols of the above formula wherein n is from about 1 to about 100.
alkyphenols of the above formula wherein n is from about 1 to about 50.
alkyphenols of the above formula wherein n is from about 9 to about 15.

Octyl Phenol 1.5 Mole Ethoxylate (i.e. n is an average of about 1.5); Octyl Phenol 5 Mole Ethoxylate; Octyl Phenol 7 Mole Ethoxylate; Octyl Phenol 9 Mole Ethoxylate; Octyl Phenol 12 Mole Ethoxylate; Octyl Phenol 40 Mole Ethoxylate; Nonyl Phenol 1.5 Mole Ethoxylate; Nonyl Phenol 4 Mole Ethoxylate; Nonyl Phenol 6 Mole Ethoxylate; Nonyl Phenol 9 Mole Ethoxylate; Nonyl Phenol 10 Mole Ethoxylate; Nonyl Phenol 10.5 Mole Ethoxylate; Nonyl Phenol 12 Mole Ethoxylate; Nonyl Phenol 15 Mole Ethoxylate; Nonyl Phenol 15 Mole Ethoxylate; Nonyl Phenol 30 Mole Ethoxylate; and Nonyl Phenol 40 Mole Ethoxylate;

### Ethoxylated Fatty Acids,

These include but are not limited to:

ethoxylates which are esterified to form either:

monoesters, i.e. RCO₂(CH₂CH₂O)ₙOH, where RCO₂H is a fatty acid; or

diesters, i.e. RCO₂(CH₂CH₂O)ₙC(═O)R.

Fatty acids include, but are not limited to:
Saturated fatty acids, which have no C═C moieties and include myristic acid, palmitic acid, stearic acid, arachidic acid, behenic acid, lignoceric acid.
Unsaturated fatty acids, including the following:
monounsaturated fatty acids, which have one C═C group such as palmitoleic acid, oleic acid, and nervonic acid;
diunsaturated fatty acids, which have two C═C groups, such as linoleic acid;
triiunsaturated fatty acids, which have three C═C groups, such as α-linolenic acid and γ-linolenic acid;
tetraunsaturated fatty acids, which have four C═C groups, such as arachidonic acid; and
pentaunsaturated fatty acids, which have five C═C groups, such as eicosapentaenoic acid.

The following may also be used:

Lauric Acid; 14 carbon fatty acids such as myristic acid; 16 carbon fatty acids such as palmitic and palmitoleic acid; 18 carbon fatty acids such as stearic acid, oleic acid, linoleic acid, α-linolenic acid, and γ-linolenic acid; 20 carbon fatty acids such as eicosapentaenoic acid; 22 carbon fatty acids such as arachidic acid; and 24 carbon carbon fatty acids such as lignoceric acid and nervonic acid.

In one embodiment, n is from about 2 to about 100.

In another embodiment, n is from about 5 to about 50.

In another embodiment, n is from about 30 to 50.

### Ethoxylated Fatty Esters or Oils (Animal & Veg.).

These are the products which result from reacting ethylene oxide with a fatty ester or an oil. When a fatty oil is used, the products is a mixture of ethoxylates of the fatty acids present in the oil, ethoxylates of glycerine, ethoxylates of mono and diglycerides, and the like.

Specific examples include, but are not limited to:

Ethoxylates of the following oils: Anise oil, Castor oil, Clove oil, Cassia oil, Cinnamon oil; Almond oil, Corn oil, Arachis oil, Cottonseed oil, Safflower oil, Maize oil, Linseed oil, Rapeseed oil, Soybean oil, Olive oil, Caraway oil, Rosemary oil, Peanut oil, Peppermint oil, Sunflower oil, Eucalyptus oil and Sesame oil; Coriander oil, Lavender oil, Citronella oil, Juniper oil, Lemon oil, Orange oil, Clary sage oil, Nutmeg oil, Tea tree oil, coconut oil, tallow oil, and lard;

In one embodiment, from 1 to about 50 moles of ethylene oxide is used per mole of the oil triglyceride.

In another embodiment, from about 30 to about 40 moles of ethylene oxide is used per mole of the oil triglyceride.

Ethylene oxide may also react with a fatty acid ester with a formula RCO₂R′ to form RCO₂(CH₂CH₂O)ₙR′. Thus, surfactants having the formula RCO₂(CH₂CH₂O)ₙR', where RCO₂H is a fatty acid and R′ is alkyl having from 1 to 6 carbons are contemplated.

One embodiment is a fatty acid methyl ester ethoxylate, wherein R′ is methyl.

In another embodiment, RCO₂H is Lauric Acid; a 14 carbon fatty acid such as myristic acid; a 16 carbon fatty acid such as palmitic and palmitoleic acid; an 18 carbon fatty acids such as stearic acid, oleic acid, linoleic acid, α-linolenic acid, and γ-linolenic acid; a 20 carbon fatty acids such as eicosapentaenoic acid; a 22 carbon fatty acids such as arachidic acid; or a 24 carbon fatty acids such as lignoceric acid and nervonic acid.

Polyethylene Glycols are ethoxylates that are unsubstituted, or terminated with oxygen on both ends, i.e. HO(CH₂CH₂O)ₙH,

### Sorbitan Derivatives:

These are ethoxylated sorbates having a fatty acid capping one or more of the ethoxylated chains. For example, polysorbate 80 has an oleate cap as shown in the structure below.

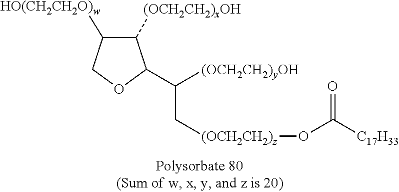

These compounds are named as POE (w+x+y+z) sorbitan mono (or di- or tri-) fatty acid.

For example, Polysorbate 80 is POE (2O) sorbitan monooleate.

Thus, the number in parenthesis is the total number of ethylene oxide units on the molecule, and the ending is the number of acid caps and the capping acid.

These include but are not limited to:

Sorbitan derivatives wherein the total number of ethylene oxide units is from 3 to 30;
Sorbitan derivatives wherein the total number of ethylene oxide units is 4, 5, or 20;
Sorbitan derivatives wherein the capping acid is laurate, palm itate, stearate, or oleate;

The sorbitan derivative may be a POE sorbitan monolaurate;

a POE sorbitan dilaurate;
a POE sorbitan trilaurate;
a POE sorbitan monopalmitate;
a POE sorbitan dipalmitate;
a POE sorbitan tripalmitate;
a POE sorbitan monostearate;
a POE sorbitan distearate;
a POE sorbitan tristearate;
a POE sorbitan monooleate;
a POE sorbitan dioleate;
or a POE sorbitan trioleate;

Specific examples include:

POE (20) sorbitan monolaurate; POE (4) sorbitan monolaurate; POE (20) sorbitan monopalmitate; POE (20) monostearate; POE (20) sorbitan monostearate; POE (4) sorbitan monostearate; POE (20) sorbitan tristearate; POE (20) sorbitan monoleate; POE (20) sorbitan 15 monoleate; POE (5) sorbitan 10 monoleate; POE (20) sorbitan trioleate; and

### Sucrose and Glucose Esters and Derivatives:

Although there are a number of sucrose and glucose based surfactants, some sucrose and glucose esters and derivatives are similar to the sorbate derivatives described above. In other words, one, several, or all of the hydroxyl moieties of the sugar are ethoxylated, and one or more of the ethoxylate chains are capped with a carboxylic acid. Other sucrose and glucose esters are simply ethoxylated, but do not have a capping carboxylic acid. Other sucrose and glucose esters may be ethoxylated and capped with an alkyl group formed by reaction with an alcohol. Other sucrose and glucose esters may be esters or ethers of the sugars with hydrophobic chains and have ethoxylates substituted in other positions on the sugar.

Various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose, and acrylates (e.g. Pemulen®).

Tonicity adjustors may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

Compositions may be aqueous solutions or emulsions, or some other acceptable liquid form. For an emulsion, one or more oils will be used to form the emulsion, and in some instances one or more surfactants will be required. Suitable oils include, but are not limited to anise oil, castor oil, clove oil, cassia oil, cinnamon oil, almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalpytus oil, sesame oil, and the like.

In one embodiment, the composition is an aqueous solution.

In another embodiment, the composition contains no ethanol.

In another embodiment, the composition contains no hyauronic acid.

In another embodiment, the composition contains no vitamin E TPGS.

In another embodiment, the composition contains no cyclodextrin A.

In another embodiment, the composition contains no cyclodextrin.

### Example I

Cyclosporin compositions may be formulated as follows.

| | Percent Ingredients | Amount needed (g) for a |
|---|---|---|
| Ingredients | (% w/v) | 1 liter batch |
| Cyclosporine | 0% for Placebo (P) | 0 grams for Placebo (P) |
| | 0.01% (A) | 0.10 (A) |
| | 0.005% (B) | 0.05 (B) |
| Carboxymethylcellulose | 0.5 | 5.0 |
| sodium | | |
| Polysorbate 80 | 1.0 | 10.0 |
| Glycerin | 1.0 | 10.0 |
| Mannitol | 0.5 | 5.0 |
| Sodium Citrate Dihydrate | 0.4 | 4.0 |
| Boric Acid | 0.25 | 2.5 |
| Sodium Borate | 0.41 | 4.1 |
| Decahydrate | | |
| Potassium Chloride | 0.14 | 1.4 |
| Purite ® | 0.010 | 0.1 |
| Purified Water | q.s. to 100% | q.s to 100% |

In one embodiment, the carboxymethylcellulose sodium is low viscosity CMC (type 7LFPH) and the Polysorbate 80 is super-refined grade.

In another embodiment, the Purite® (preservative) is omitted, and the solution is packaged in single-use vials.

Compositions P, A, and B are prepared according to the following procedure.

1. Measure Purified Water to about 90% of the batch size and place in an appropriate beaker or container.
2. Begin mixing the water with a strong mixer (Rotosolver®) to obtain a strong vortex.
3. Add the pre-weighed carboxymethylcellulose sodium into the strong vortex. Continue strong mixing for at least 1 hour.
4. Slow mixer to a slow speed.
5. Add and dissolve the pre-weighed polysorbate 80.
6. Add and dissolve the pre-weighed glycerin.
7. Add and dissolve the pre-weighed mannitol.
8. Add and dissolve the pre-weighed sodium citrate dehydrate.
9. Add and dissolve the pre-weighed boric acid.
10. Add and dissolve the pre-weighed sodium borate decahydrate.
11. Add and dissolve the pre-weighed potassium chloride.
12. Check pH and adjust if necessary. Target pH is 7.5+/−0.1.
13. Add and dissolve the pre-weighed Purite.
14. Add sufficient quantity of Purified Water to attain the final batch volume. This will provide the finished placebo formulation (P).
Procedure for either 0.01% (A) or 0.005% (B)
15. Measure the exact amount of placebo needed to satisfy the batch size requirements and place in a media bottle that contains a magnetic stir bar.
16. Add and dissolve the pre-weighed cyclosporine. Stir at a slow speed to avoid foaming. It will usually take overnight mixing to completely dissolve the cyclosporine.
17. After overnight mixing is completed, pump the cyclosporine solution through a Millipore Milligard® pre-filter and a Pall Suporlife® sterilizing filter and collect the filtrate aseptically.
18. The sterile filtrate can then be aseptically dispensed into multidose dropper bottles suitable for ophthalmic purpose.
19. The finished product should be tested for cyclosporine assay, pH, osmolality, viscosity, Purite®, sterility, and antimicrobial effectiveness.
20. The finished product should be store at room temperature and protected from light.

### Example II

In another embodiment, cyclosporin compositions are formulated as follows. All values are w/v % unless stated otherwise.

| | Composition | Composition | Composition |
|---|---|---|---|
| Ingredients | C | D | E |
| | Aqueous | Aqueous | Emulsion |
| | Solution | Solution | |
| Cyclosporine A | 0.01 | 0.005 | 0.01 |
| Purite | 0.01% (100 | 0.01% (100 | 0.0% (0 |
| | ppm) | ppm) | ppm) |
| Polysorbate 80 | 1.0 | 1.0 | 1.0 |
| Glycerin | 1.0 | 1.0 | 2.2 |
| Mannitol | 0.5 | 0.5 | N/A |
| Sodium | 0.5 | 0.5 | N/A |
| Carboxymethylcellulose | | | |
| (CMC) - 7LFPH | | | |
| Sodium Citrate | 0.4 | 0.4 | N/A |
| Dihydrate | | | |
| Boric Acid | 0.25 | 0.25 | N/A |
| Sodium Borate | 0.41 | 0.41 | N/A |
| Decahydrate | | | |
| Potassium Chloride | 0.14 | 0.14 | N/A |
| Castor Oil | N/A | N/A | 1.25 |
| Pemulen TR-2 | N/A | N/A | 0.05 |
| Sodium Hydroxide | N/A | N/A | pH 7.4 |
| Purified Water | QS | QS | N/A |

### Administration

The cyclosporin compositions of the invention may be administered to treat a patient having allergic conjunctivitis. To “treat,” as used here, means to deal with medically. It includes administering the compositions of the invention to prevent atopic keratoconjunctivitis as well as to lessen its severity. The cyclosporin compositions may be administered once, twice, three times, four times, or more, daily.

### Example III

48 male beagle dogs were divided into four study groups and administered a solution containing 0.01% cyclosporin A topically to each eye. Groups 1 and 2 received a single dose for ten days; group 1 received cyclosporin A 0.01%, and group 2 received cyclosporin A 0.05% (Restasis®). Groups 3 and 4 received two doses per day for ten days; group 3 received cyclosporin A 0.01%, and group 4 received cyclosporin A 0.05% (Restasis®). Ocular tissue was examined at 12 hours post dose on day 1 in study groups 1 and 2, and at 0.5, 2, 4, 6, and 12 hours post dose on day 10 in study groups 3 and 4.

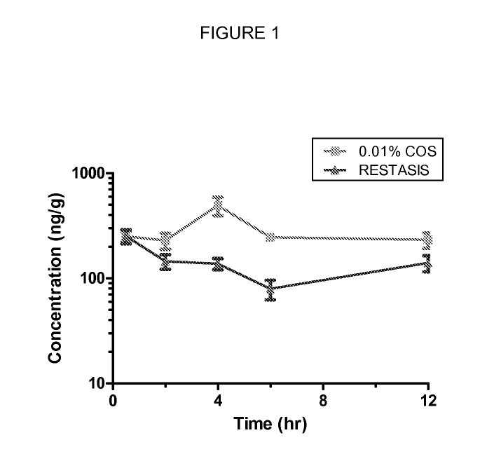

FIG. 1 shows the mean concentration of cyclosporin in the cornea after 10 days BID administration. The 0.01% cyclosporin A solution achieves an approximately 2-fold increase in cyclosporin exposure as compared to Restasis.

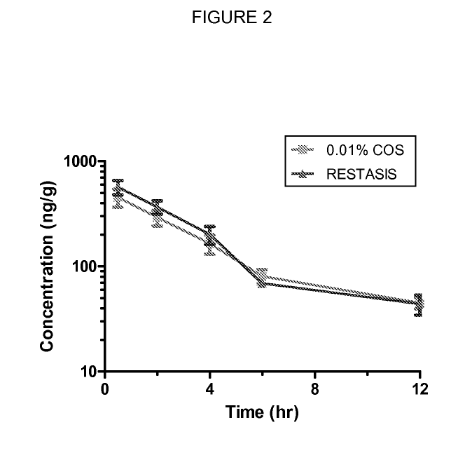

FIG. 2 shows the mean concentration of cyclosporin in the palpebral conjunctiva after 10 days BID administration. AUC values are within approximately 20% following administration of either 0.01% cyclosporin A and Restasis®.

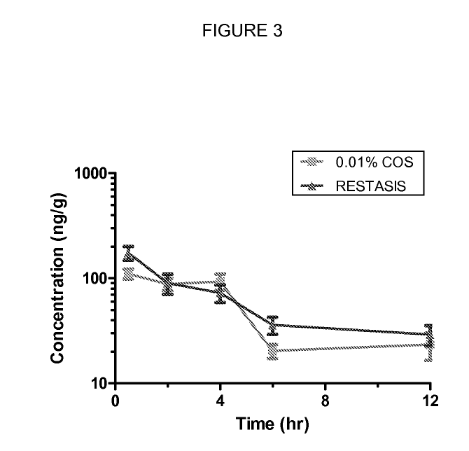

FIG. 3 shows the mean concentration of cyclosporin in the bulbar conjunctiva after 10 days BID administration. AUC values are within approximately 20% following administration of either 0.01% cyclosporin A and Restasis®.

Table 1 shows PK parameters in ocular tissues following 10 days bid administration in study groups 3 and 4.

| | | | RESTASIS ® | | Relative |
|---|---|---|---|---|---|
| Mean | 0.01% COS | | (0.05% CsA) | | % F |
| Tissue | Cmax | AUC0-12 | Cmax | AUC0-12 | Based on |
| Levels | (ng/g) | (ng · hr/g) | (ng/g) | (ng · hr/g) | AUC |
| Cornea | 493 | 3250 | 251 | 1520 | 218 |
| Palpebral | 454 | 1640 | 567 | 2020 | 86.6 |
| Conjunctiva | | | | | |
| Bulbar | 110 | 574 | 175 | 709 | 84.9 |
| Conjunctiva | | | | | |

The foregoing data show that cyclosporine A 0.01% achieves comparable levels of drug exposure in each of the cornea and conjunctiva following a single dose or BID for 10 days.

The invention may be described as follows:

1. A method of treating an allergic conjunctivitis, the method comprising topically administering to an eye affected with such a condition a composition comprising cyclosporin, at a concentration between about 0.001% (w/v) to about 0.01% (w/v), to an eye of a subject having allergic conjunctivitis.
2. The method of 1, wherein the allergic conjunctivitis is seasonal allergic conjunctivitis, perennial allergic conjunctivitis, atopic keratoconjunctivitis, or vernal keratoconjunctivitis.
3. The method of 1 or 2, wherein the cyclosporin is cyclosporin A.
4. The method of 1, 2, or 3, wherein the cyclosporin A is administered at a concentration of between about 0.005% (w/v) and about 0.01% (w/v).
5. The method of 1, 2, or 3, wherein the cyclosporin A is administered at a concentration of about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), or about 0.001% (w/v).
6. A method of treating atopic keratoconjunctivitis, the method comprising the step of topically administering to an eye affected with such a condition a composition comprising cyclosporin A at a concentration of 0.005% (w/v) or 0.001% (w/v).
7. A method of treating vernal keratoconjunctivitis, the method comprising the step of topically administering to an eye affected with such a condition to a patient having atopic keratoconjunctivitis a composition comprising cyclosporin A at a concentration of 0.005% (w/v) or 0.001°)/0 (w/v).
8. The method of any of the foregoing, wherein the composition further comprises

Carboxymethylcellulose sodium, 0.5% (w/v),

Polysorbate 80, 1.0% (w/v),

Glycerin, 1.0% (w/v),

Mannitol, 0.5% (w/v),

Sodium citrate dihydrate, 0.4% (w/v),

Boric acid, 0.25% (w/v),

Sodium borate decahydrate, 0.41% (w/v),

Potassium chloride 0.14 0.41% (w/v), and, optionally, Purite® (i.e., stabilized chlorine dioxide).

9. The method according to any of the foregoing, wherein 25-50 μg of the cyclosporin composition is administered to the eye.
10. The method of 9, wherein the cyclosporin composition is administered one, two, three, or four times a day.

What is claimed is:

1. A method of treating an allergic conjunctivitis, the method comprising topically administering to an eye affected with such a condition a composition comprising cyclosporin A, at a concentration between about 0.001% (w/v) to about 0.01% (w/v), to an eye of a subject having allergic conjunctivitis.

2. The method of 1, wherein the allergic conjunctivitis is selected from the group consisting of seasonal allergic conjunctivitis, perennial allergic conjunctivitis, atopic keratoconjunctivitis, and vernal keratoconjunctivitis.

3. A method of treating atopic keratoconjunctivitis, the method comprising the step of topically administering to an eye affected with such a condition a composition comprising cyclosporin A at a concentration of 0.005% (w/v) or 0.001% (w/v).

4. The method of claim 3, wherein the composition further comprises
Carboxymethylcellulose sodium, 0.5% (w/v),
Polysorbate 80, 1.0% (w/v),
Glycerin, 1.0% (w/v),
Mannitol, 0.5% (w/v),
Sodium citrate dihydrate, 0.4% (w/v),
Boric acid, 0.25% (w/v),
Sodium borate decahydrate, 0.41% (w/v), and
Potassium chloride 0.14% (w/v).

5. A method of treating vernal keratoconjunctivitis, the method comprising the step of topically administering to an eye affected with such a condition to a patient having atopic keratoconjunctivitis a composition comprising cyclosporin A at a concentration of 0.005% (w/v) or 0.001% (w/v).

6. The method of claim 5, wherein the composition further comprises
Carboxymethylcellulose sodium, 0.5% (w/v),
Polysorbate 80, 1.0% (w/v),
Glycerin, 1.0% (w/v),
Mannitol, 0.5% (w/v),
Sodium citrate dihydrate, 0.4% (w/v),
Boric acid, 0.25% (w/v),
Sodium borate decahydrate, 0.41% (w/v), and
Potassium chloride 0.14% (w/v).
